(19) United States
(12) Patent Application Publication
(10) Pub. No.: US 2015/0086516 A1
(43) Pub. Date: Mar. 26, 2015

(54) Methods, Compositions, and Devices to Induce Mobilization and Recruitment of Progenitor Cells

(71) Applicant: Massachusetts Institute of Technology, Cambridge, MA (US)

(72) Inventors: Laura Indolfi, Boston, MA (US); Elazer R. Edelman, Brookline, MA (US)

(21) Appl. No.: 14/496,755

(22) Filed: Sep. 25, 2014

Related U.S. Application Data
(60) Provisional application No. 61/882,176, filed on Sep. 25, 2013.

(51) Int. Cl. A61K 35/44; C12N 11/02; C12N 5/071

(52) U.S. Cl. CPC A61K 35/44; C12N 5/069; C12N 11/02
USPC 424/93.7; 435/177

(57) ABSTRACT

Methods, compositions and systems are provided for mobilizing and recruiting progenitor, progenitor-like cells, and/or stem cells to a target site, such as an in vivo site in a patient in need of treatment. In embodiments, the methods include embedding cells in a matrix material having a three-dimensional structure capable of contacting the cells in a non-planar manner to alter the secretome of the cells and/or miRNA expression in a manner effective to recruit progenitor, progenitor-like, or stem cells.

### CROSS-REFERENCE TO RELATED APPLICATION

This application claims priority to U.S. Provisional Patent Application No. 61/882,176, filed Sep. 25, 2013, which is incorporated herein by reference.

### BACKGROUND

Endothelial progenitor cells (EPCs) are bone marrow-derived cells that can be found in the peripheral and umbilical cord blood. EPCs have the ability to differentiate into multiple cell lines. Endogenous factors like cytokines and growth factors mediate recruitment of EPCs into the circulation.

Circulating EPCs have a wide array of functions in tissue regeneration, tissue remodeling, and cancer progression. For example, in tumors and ischemic tissues, EPCs have a direct structural role of differentiating into mature endothelial cells, and an indirect paracrine effect by secreting angiogenic factors. Therefore, EPCs are being studied in various diseases ranging from ischemia, diabetic retinopathy, and cancer.

The discovery that these cells can be mobilized from their bone marrow niche to sites of inflammation and tumors to induce neovasculogenesis has afforded an opportunity to develop cutting edge therapies. Strategies utilizing injections of single active agents to induce recruitment of EPCs in the circulating blood, however, have shown only marginal success. These failures have been attributed in part to the selective use of individual agents and/or their poor time residency at the site of injection. Strategies that overcome one or more of these disadvantages are desirable.

### BRIEF SUMMARY

Provided herein are methods of mobilizing and recruiting progenitor, progenitor-like cells, and/or stem cells to a target site. Also provided are methods of treatment, devices, and compositions. The methods and devices provided herein have excellent time residency at the site of deployment, and rely on cells embedded in a matrix material instead of individual agents to recruit progenitor, progenitor-like, and/or stem cells.

In embodiments, the methods comprise embedding cells in a matrix material having a three-dimensional structure which contacts the cells in a non-planar manner to alter the expression of one or more miRNAs in a manner effective to recruit progenitor, progenitor-like, or stem cells.

In embodiments, the methods comprise embedding cells in a matrix material having a three-dimensional structure which contacts the cells in a non-planar manner to induce a secretome in an amount effective to recruit progenitor, progenitor-like, or stem cells, wherein the secretome comprises at least one of (i) one or more factors, or (ii) one or more miRNAs.

In embodiments, the methods comprise implanting matrix embedded cells at or near a target site, wherein the matrix embedded cells release at least one of (i) one or more factors, or (ii) one or more miRNAs in an amount effective to recruit progenitor, progenitor-like, or stem cells.

In embodiments, the methods comprise selecting one or more target sites within the body of a patient in need of treatment; and implanting matrix embedded cells at or near the one or more target sites, wherein the matrix embedded cells release at least one of (i) one or more factors, or (ii) one or more miRNAs in an amount effective to recruit progenitor, progenitor-like, or stem cells to the one or more target sites.

In embodiments, devices are provided for use in mobilizing and recruiting progenitor, progenitor-like, or stem cells. The devices, in some embodiments, comprise a biocompatible matrix material having a three-dimensional structure; and cells embedded within the three-dimensional structure of the matrix material, wherein the cells and matrix material are configured to release at least one of (i) one or more factors, or (ii) one or more miRNAs in an amount effective to recruit progenitor, progenitor-like, or stem cells, wherein the device is in a form suitable for insertion at a target site within a patient in need thereof, the target site comprising a tumor or other damaged or diseased tissue in the patient.

### BRIEF DESCRIPTION OF THE DRAWINGS

FIG. 1 depicts the release of growth factors by endothelial cells in two-dimensional and three-dimensional environments.

FIG. 2 depicts the release of growth factors by endothelial cells in two-dimensional and three-dimensional environments.

FIG. 3 depicts the number of endothelial progenitor cells recruited by the factors released by endothelial cells in two-dimensional and three-dimensional environments.

FIG. 4 depicts the number of endothelial progenitor cells recruited by the factors released by endothelial cells in two-dimensional and three-dimensional environments.

FIG. 5 depicts the number of endothelial progenitor cells recruited after intraperitoneal injection.

FIG. 6 shows the raw data collected during experiments measuring the number of endothelial progenitor cells recruited after intraperitoneal injection.

FIG. 7 depicts the increase of miRNA-126 expression of endothelial cells in three-dimensional environments compared to a two-dimensional setting.

FIG. 8 is a Principal Component Analysis of endothelial cells' miRNAs in two-dimensional and three-dimensional environments.

FIG. 9A-1 depicts the clustering observed for the top 50 miRNAs from cells in a two-dimensional environment.

FIG. 9A-2 depicts the clustering observed for the top 50 miRNAs from cells in a three-dimensional environment.

FIG. 9B is an expanded view of a portion of FIG. 9A-2.

FIG. 10 is a volcano plot showing the relation between the logarithm of the p-values and the log fold change between endothelial cells in two-dimensional and three-dimensional environments.

FIG. 11 is a laser speck image that tracked perfusion of the tested mice.

FIG. 12 compares the percent recovery observed from endothelial cells in two-dimensional and three-dimensional environments.

### DETAILED DESCRIPTION

Provided herein are methods of mobilizing and recruiting progenitor, progenitor-like cells, and/or stem cells to a target site. Also provided herein are matrix materials containing embedded cells that can induce mobilization of various cells, such as stem and/or progenitor cells, and recruit them to a target site. The target site may be within a human or other mammal.

The matrix materials are three-dimensional structures that contact the embedded cells in a non-planar manner. The non-planar morphology of the matrix material, as opposed to a planar two-dimensional morphology, imparts the cells with a non-planar configuration that at least partially replicates the configuration that the cells would have in an in vivo environment.

As a result, in embodiments, the secretome of the embedded cells is affected in a manner that leads to the recruitment of progenitor, progenitor-like cells, and/or stem cells to a target site. In one embodiment, the release from the embedded cells of one or more factors that mediate recruitment of EPCs into the circulation, such as cytokines and growth factors, is altered. In another embodiment, the release from the embedded cells of one or more miRNAs that mediate recruitment of EPCs into the circulation is altered. In a further embodiment, the release from the embedded cells of (1) one or more factors that mediate recruitment of EPCs into the circulation is altered, and (2) one or more miRNAs that mediate recruitment of EPCs into the circulation is altered. The term “altered”, as used herein regarding a secretome, means that a secretome is changed in a way that leads to the recruitment of progenitor, progenitor-like, and/or stem cells. In some embodiments, the release of one or more factors and/or one or more miRNAs is upregulated. In other embodiments, the release of one or more factors and/or one or more miRNAs is downregulated. In still further embodiments, the release of one or more factors is upregulated and the release of one or more miRNAs is downregulated, or vice versa.

Furthermore, in embodiments, embedding the cells in a matrix material alters the expression of one or more miRNAs within the cells in a manner that leads to the recruitment of progenitor, progenitor-like cells, and/or stem cells. Not wishing to be bound by any particular theory, it is believed that this occurs when the expression of those miRNAs involved in recruiting progenitor, progenitor-like, and/or stem cells to a target site is affected. miRNAs generally are part of the RNA of cells that affect gene expression.

Therefore, in one embodiment, embedding the cells in the matrix materials provided herein affects the cells in at least one of the following ways: (1) the release from the embedded cells of one or more factors that mediate recruitment of EPCs into the circulation is altered, (2) the release from the embedded cells of one or more miRNAs that mediate recruitment of EPCs into the circulation is altered, or (3) the expression of one or more miRNAs involved in recruiting EPCs is altered in a manner that leads to the recruitment of progenitor, progenitor-like, and/or stem cells.

Not wishing to be bound by any particular theory, it is believed that when a cell is embedded in a matrix, the microarchitecture of the matrix's substratum imposes a specific cytoskeleton configuration that may alter the internal tensional state of the cells. This alteration may interfere with the embedded cells' signaling pathways, thereby altering the release of factors, the release of miRNAs, and/or miRNA expression.

These changes can modulate the embedded cells' regulation of vascular disease through the mobilization and recruitment of progenitor, progenitor-like cells, and/or stem cells. The embedded cells can be used to mobilize and recruit progenitor, progenitor-like cells, and/or stem cells in vitro or in vivo.

### Embedded Cells

In one embodiment, the embedded cells include endothelial cells, endothelial-like cells, analogs thereof, or a combination thereof. In one embodiment, the embedded cells are, or are grown from, human aortic endothelial cells and/or human umbilical vein endothelial cells. The embedded cells may be collected from donors and grown in an endothelial grown medium, which may be supplemented.

As used herein, the term “embedded” means anchored and/or associated with. Therefore, in some embodiments, the embedded cells are anchored and/or physically associated with the matrix materials. Embedding the cells in the matrix material permits the matrix material to contact the cells in a non-planar manner, thereby imparting the cells with a non-planar configuration. Endothelial cell anchorage to the matrix materials may be mediated through the interactions of integrins and focal adhesion complexes (FAC) incorporating proteins such as vinculin.

The embedded cells of the matrix materials can modulate recruitment of various cells, including progenitor, progenitor-like cells, and stem cells. In one embodiment, the embedded cells of the matrix materials attain a configuration unique to their environment with a secretome that optimizes recruitment of various cells, including EPCs. This may arise from the tailored release of any of a number of factors, including but not limited to, SDF-1, HGF, PDGF, TIMP-1, and TIMP-2, which can have a paracrine effect on the targeted cell type. Moreover, the release or expression of certain miRNAs may be altered, including, but not limited to, those believed to be involved in recruiting progenitor, progenitor-like cell, and/or stem cells, such as miR-210, miR-126, miR-222, or a combination thereof. The miRNAs that are present within the cells can be release through exosomes. Upon their release, the miRNAs can affect adjacent cells.

Matrix Materials

In embodiments, the matrix materials that host the embedded cells form a three-dimensional, biocompatible matrix. The matrix materials may be polymeric materials. In one embodiment, the biocompatible matrix is a three-dimensional matrix of denatured collagen. For example, the biocompatible matrix may be a porous collagen scaffold (Gelfoam®, NY, N.Y.).

Other biocompatible natural or synthetic polymeric matrix materials are envisioned.

The three-dimensional, biocompatible matrix may include pores, struts, or a combination thereof. In some embodiments, the biocompatible matrix has pores that range in size from about 1 μm to about 2000 μm. The biocompatible matrix, in one embodiment, has pores that range in size from about 5 μm to about 150 μm. The biocompatible matrix, in another embodiment, has pores that range in size from about 50 μm to about 150 μm. The size of the pores may be determined by the procedure described in the Examples below.

Within the biocompatible matrix, the single struts may have dimensions ranging from about 0.01 μm to about 500 μm. In certain embodiments, the single struts have dimensions ranging from about 10 μm to about 50 μm.

Not wishing to be bound by any particular theory, it is believed that, in some embodiments, the size correspondence between the embedded cells and the strut diameter within the biocompatible matrix induces the embedded cells to achieve a non-planar morphology. This may determine changes in actin fiber organization, and may depend strongly on local substratum patterning. The embedded cells may circumferentially wrap around the struts having dimensions smaller than the cells, causing the actin filaments to become oriented perpendicularly to the long direction of the strut, and parallel to the direction of bending. As strut dimensions rise and exceed the dimensions of the embedded cells, the cells may align their actin filaments increasingly parallel to the longest aspect of the strut. Therefore, actin fibers may be oriented parallel to the major direction of curvature and never haphazardly arranged, thus determining one major filament direction and two nodes at the polar extremes of the cells consistent with a “gripping” nature of embedded cells.

In one embodiment, the matrix materials enable the retention and transport of embedded cells in a unit dose that remain viable for a desired time.

The matrix materials may contain one or more biocompatible additives. For example, the additives may include a pharmaceutically active ingredient.

Devices for use in the methods described herein include the matrix material and the embedded cells in a suitable form. The suitable form may be of a size and geometry effective for deployment into and retention at a target site in a patient. Accordingly, the particular form may vary depending on the target site location, desired coverage area, handling and deployment considerations, and the mechanical properties of the matrix material. The device may be provided in various forms, including sheets (e.g., a flexible wrap); gels, or particulates, with or without a housing structure, which may itself have apertures and/or be porous, and which may assist in handling, deployment, or in vivo retention of the matrix material with embedded cells.

Generally, the recipient, or patient, receives the embedded cells by implanting or arranging the matrix materials containing the embedded cells at or near the target site. The recipient may be a human or other mammalian animal in need of treatment.

The target site may be or include a tumor site, a site of injury, a site of disease, or a combination thereof. The matrix materials containing embedded cells may be implanted or arranged at or near the target site. In one embodiment, the matrix materials containing embedded cells are implanted perivascularly to the target tissue. In another embodiment, the recipient is provided an implantable matrix material that hosts embedded cells selected from endothelial cells, endothelial-like cells, analogs thereof, or a combination thereof. The matrix materials and embedded cells may be provided to the recipient in an amount effective to recruit various cells, including progenitor, progenitor-like cells, and stem cells, in the recipient. In some embodiments, the amount is effective to induce mobilization and recruitment of progenitor, progenitor-like, and/or stem cells to the target site. In other embodiments, the amount is effective to modulate the recruitment of progenitor, progenitor-like, and/or stem cells, especially in patients with disease conditions that extend beyond specific tissues.

In one embodiment the matrix materials and embedded cells can be provided in the vicinity of an injury prior to, coincident with, or subsequent to another intervention, e.g., pharmacologic, mechanical, or other cell-based implants.

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

### EXAMPLES

The materials and methods described herein have been tested using in vitro and in vivo models. All materials were purchased from Sigma Co. unless otherwise specified.

### Example 1

### Recruitment of EPCs

The role of endothelial cells (ECs) embedded in a three-dimensional biocompatible matrix to act as paracrine recruiters of vascular stem cell precursors like EPCs was investigated. The matrix-embedded endothelial cells (MEECs) were used as a therapeutic device to locally recruit vascular stems cells to a target site, which, in this example, was a site of injury.

Conditioned media from MEECs was used to test their ability to recruit EPCs through specific secreted factors. Conditioned media was gathered from cells in 2D or 3D cultures and secretome levels were measured by multiplex ELISA. ECs were cultured on three-dimensional matrices for 14 days to allow confluence and full invasion of the device.

Human aortic endothelial cells pooled from three donors were grown in endothelial growth medium supplemented with EGM-2 growth supplements (Lonza). ECs were grown on gelatin-coated tissue culture plates (two-dimensional ECs)(0.1% gelatin type A, Sigma, St. Louis, Mo.) or in three-dimensional gelatin matrices (MEECs)(Gelfoam®, Pfizer, New York, N.Y.).

For cell-matrix engraftment, compressed sponges were cut into 1×1×0.3 cm blocks and hydrated in culture medium at 37° C. for ≧4 hours. Then 4.5×10⁴ ECs (suspended in ˜50 μL media) were seeded onto one surface of the hydrated matrix, allowed 1.5 hour to attach before turning the matrix over and seeding an additional 4.5×10⁴ in growth media. After a further 1.5 hours of incubation, each piece was added to a separate 30 mL polypropylene tube containing 10 mL of culture medium. Matrices were cultured for up to 3 weeks, with media changed every 48-72 hours, under standard culture conditions (37° C. humidified environment with 5% CO₂).

For this example, three-dimensional matrices of denatured collagen (Gelfoam®, Pfizer, New York, N.Y.) were used as the matrix materials. Morphological characterization of the matrices was carried out using an environmental Scanning Electron Microscope (eSEM; Philips/FEI XL30 FEG-SEM). The matrices were visualized in their hydrated state using low vacuum settings to preserve the architecture of the scaffolds. Porosity of the matrix materials was established through serial cryo sectioning of the matrix in 40 μm slices and subsequent staining with Biebrich's Scarlet Acid Fuchsin dye (IMEB). Images were taken using a Nikon epifluorescence microscope (inverted Eclipse Ti-E, Nikon) and processed using ImageJ software to determine the maximum diameter of the pores.

Conditioned media from MEECs or ECs on a standard two-dimensional petri dish was collected and used for a migration assay. EPCs were seeded on the top membrane of an insert-well and were allowed to adhere for an hour. The bottom of the well was then filled with the conditioned media and incubated for 24 hours. Then the number of EPCs that migrated in the media was analyzed by cell counter. The results show that conditioned media of MEECs was four times more powerful than ECs in a conventional two-dimensional setting at recruiting EPCs through the insert.

All of the EPCs seeded on the insert migrated in the bottom-well in the case of media from MEECs. The possibility that matrix degradation could be implied in the process by releasing proteolytic products able to recruit EPCs was discarded by testing conditioned media from devices without cells at different time points. This analysis provided results that were comparable with control media alone and ECs in a two-dimensional petri dish.

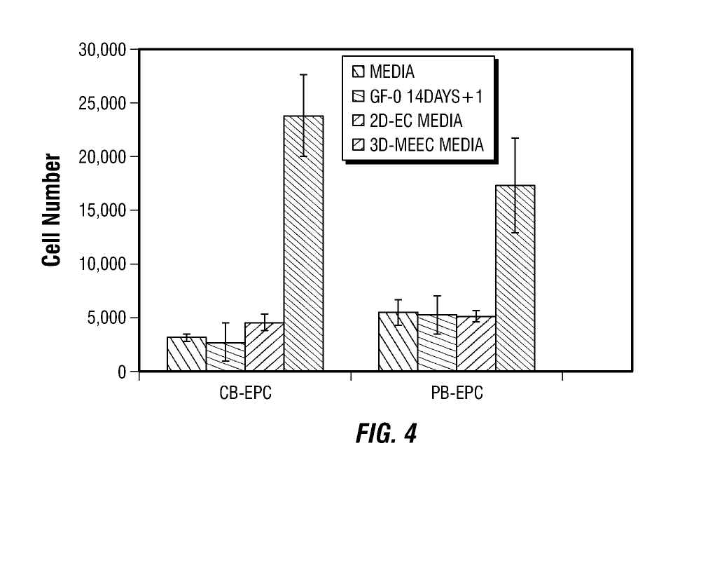

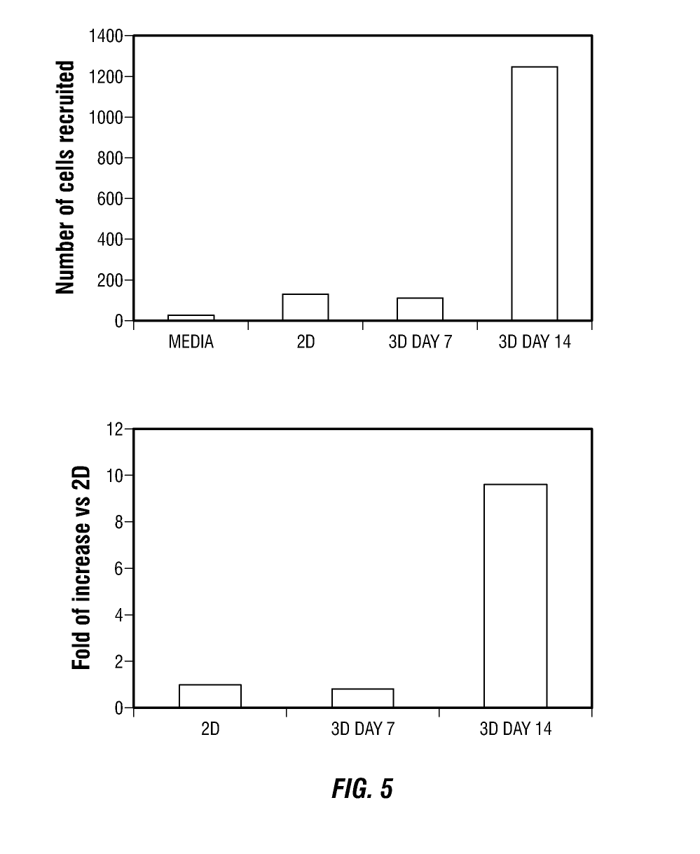

The ability of the MEECs to attract EPCs was tested by seeding an insert with 20,000 EPCs and letting them adhere for 1 hour. Specifically, paracrine effect of secretome was tested by exposing cord blood (CB) or peripheral blood (PC)-EPCs seeded on the top membrane of a transwell plate to CM from 3D-EC and 2D-EC. Conditioned media was then added in the bottom well of the insert and incubated for 24 hours. After the 24 hour incubation period, the number of cells that migrated through the membrane toward the conditioned media was evaluated. FIG. 3 and FIG. 4 show the factors released by MEECs recruited EPCs in these in vitro experiments. The totality of EPCs migrated to the bottom of the insert in the presence of conditioned media form gelfoam. The in vitro migration assay (FIG. 4) clearly demonstrated the paracrine effect of the secreted factors, which resulted in a full migration of cells toward the bottom well when in the presence of MEEC conditioned media. As shown at FIG. 4, the factors secreted from the MEECs were able to paracrine recruit all of the EPCs, regardless of their tissue of extraction.

### Example 2

### Morphology of MEECs

The morphology of the two-dimensional ECs and the MEECs was visualized by eSEM. Cells were fixed in 4% paraformaldehyde overnight and counterstained for 30 minutes with 0.5% uranyl acetate solution to increase visibility under microscope. Samples were then analyzed using a back scatter mode in low vacuum environment. For immunofluorescence analysis, cells were fixed in 4% paraformaldehyde, EC-engrafted matrices were additionally incubated in 30% sucrose, frozen, and cryo sectioned in 40 μm slides. The cytoskeleton was visualized by staining cells for filamentous actin using fluorescent-phalloidin. Cells were exposed to 0.2 M glycine for 10 minutes and incubated with 0.2% triton X-100 in phosphate buffered saline for 10 minutes. Goat serum (4%) in phosphate buffered saline with 1% bovine serum albumin was applied for 1 hour at room temperatures (RT). Vinculin primary antibody (1:50, Santa Cruz, Santa Cruz, Calif.) was applied to the cells overnight at 4° C. Secondary antibody, alexa fluoro 488 (1:50, Invitrogen, Carlsbad, Calif.), was applied to the cells for 1 hour at RT with or without rhodamine-phalloidin (1:250, Invitrogen) for visualization of F-actin. Cells were mounted with VectaShield containing DAPI (Vector Labs, Burlingame, Calif.). Imaging was performed via confocal microscopy (Zeiss LSM510, Germany, Confocal Core Facility at the Beth Israel Deaconess Medical Center, Harvard Medical School, Boston, Mass.). In the case of MEECs, multiple z-stack imaging was carried out due to cells lying on different focal planes; three-dimensional rendering of the raw data was then performed using the confocal image analysis software. Quantification of vinculin inside the cell was carried out by fluorescence intensity analysis. Single cell area was selected and the intensity levels of the green channel were detected using the confocal image analysis software. This process was repeated for each condition of cell culture (standard and Src-inhibited 2D and 3D settings, n=20).

Images of fluorescently labeled ECs in a two-dimensional setting and MEECs were analyzed to determine the orientation of actin filaments. Individual cells were selected and the value of the angle θ, defined as the angle between each actin filaments and the major axis of the cell, was evaluated. To normalize the results, the data were represented in terms of cosine of θ, spanning between 0 (filaments orthogonal to the axis direction) and 1 (filaments parallel to the axis direction). To have additional quantitative analysis on cytoskeleton remodeling, the lattice defined by the active filaments was determined highlighting the directions achieved by the filaments together with the number of nodes, i.e., point of connection between two filaments, in each cell. It is worth to noting that analysis of ECs within matrices was performed with the help of the 3D rendering of the z-stack data to reduce misjudgments due to planar projection of the different focal planes.

### Example 3

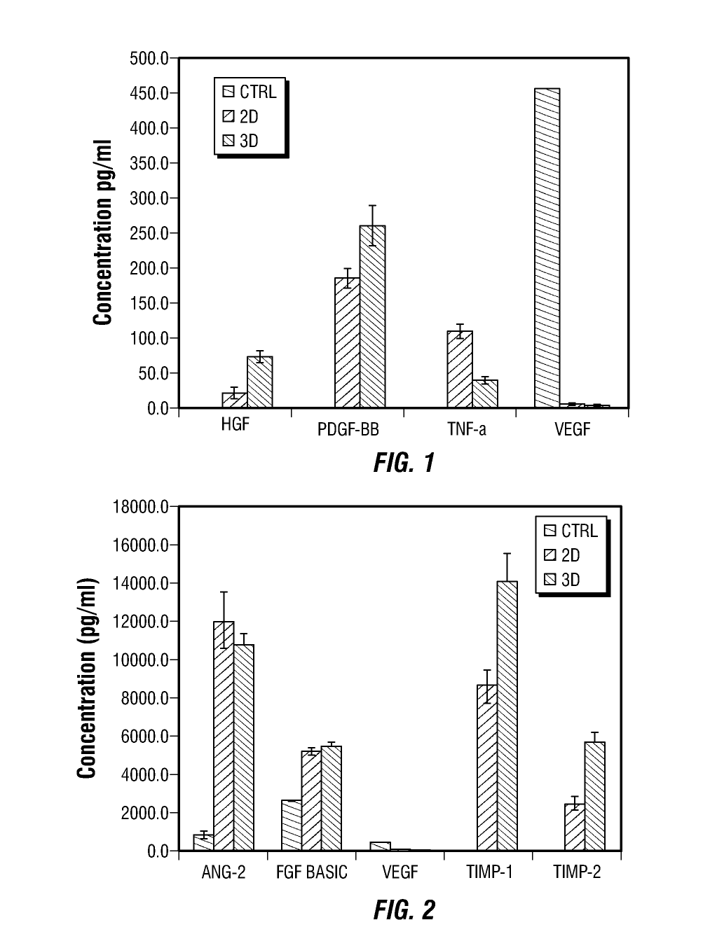

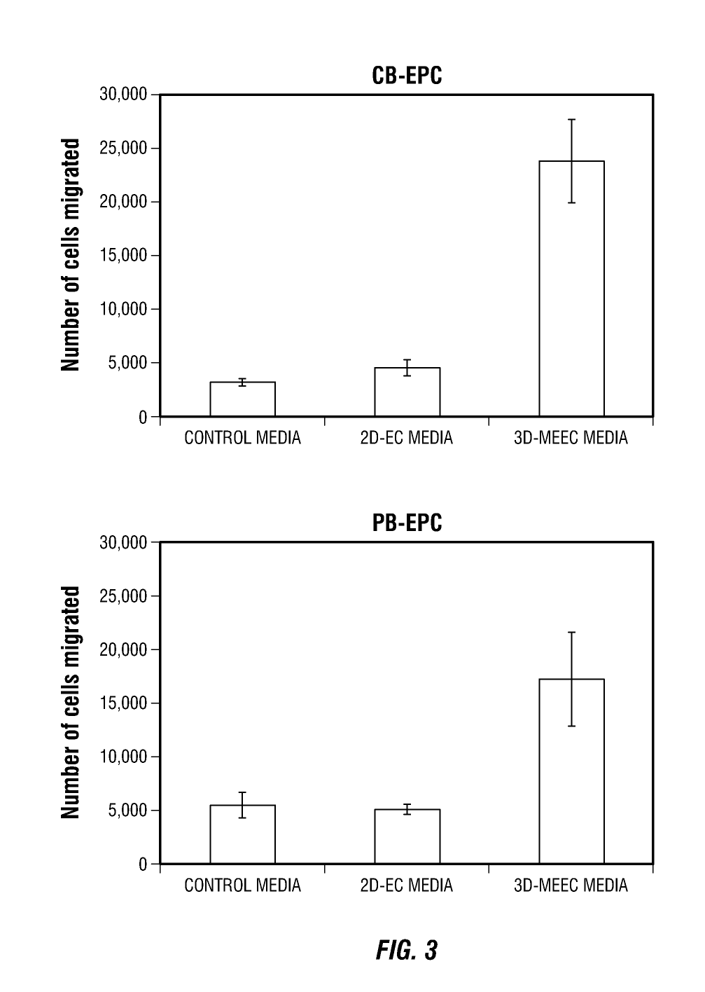

The secretomes of the MEECs and the ECs in a two-dimensional setting of Example 1 were screened (see FIG. 1 and FIG. 2). In addition to morphological differences, secretome of MEECs differed from ECs in a two-dimensional setting. Release of active agents known to be involved in EPC recruitment, such as HGF and PDGF-BB, was up-regulated in MEECs compared to ECs in a two-dimensional setting. FIG. 1 depicts how the released concentration of HGF and PDGF-BB growth factors increased in MEECs compared to ECs in a two-dimensional setting, while TNF-a levels were reduced. Therefore, simultaneously, inflammatory-inducing factors were down-regulated with the 2-fold lower release of TNF-a.

Specific attention was paid to the factors that mobilize and recruit EPCs. The enzyme-linked immunosorbent assay (ELISA) revealed that the MEECs upregulated the secretion of several growth factors, including HGF and PDGF-BB. The MEECs secreted 3 times more HGF and 1.5 times more PDGF-BB than the ECs in a two dimensional setting. The different secretion profiles demonstrated that MEECs have the potential to control and tune EPC mobilization and recruitment of EPCs.

### Example 4

An experiment similar to the one described in Example 1 was conducted to test the recruitment of EPCs in vivo. Conditioned media (2 mL) from 3D-EC and 2D-EC was injected into the peritoneal cavity of black mice. After 24 hours, peritoneal lavage was performed and extracted cells were analyzed by three-markers FACS. EPCs were determined by CD45⁻, CD34⁺, and Flk-¹⁺ cells.

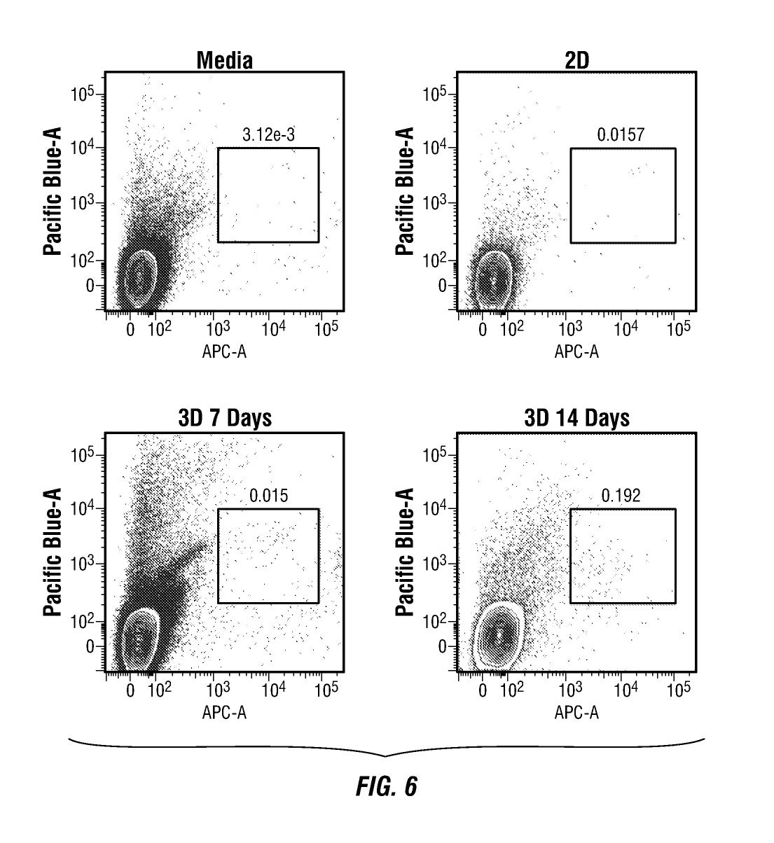

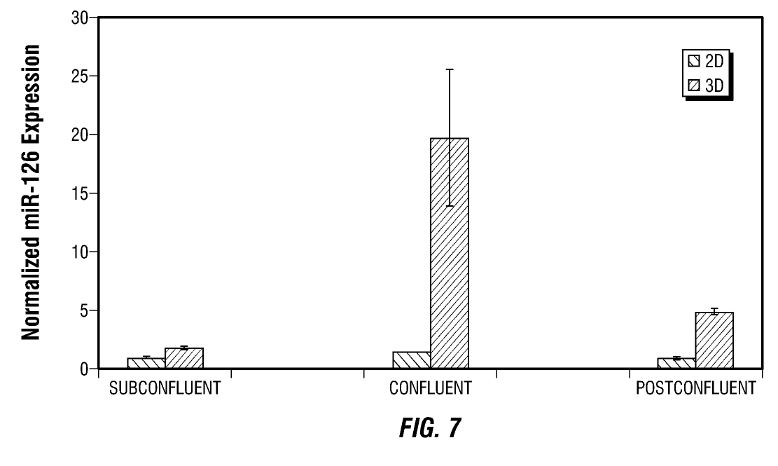

A reading was then taken of the EPCs recruited at the site 24 hours after injection (see FIG. 5 and FIG. 6). As shown at FIG. 5A, injection of conditioned media from MEECs cultured for 14 days resulted in a 9-fold increase in the in vivo mobilization of EPC to the site of injection in healthy mice when compared to ECs in two-dimensional setting.

In a separate study, hind limb ischemia was induced in mice using femoral artery ligation. Conditioned media from 2D or 3D cultures was delivered to the ischemic limb using local delivery from alginate beads. The extent of recovery of perfusion and neovascularization was quantified using laser speckle imaging and histological analysis.

The injection also resulted in slightly improved blood flow in ischemic mice after 5 days of treatment.

### Example 5

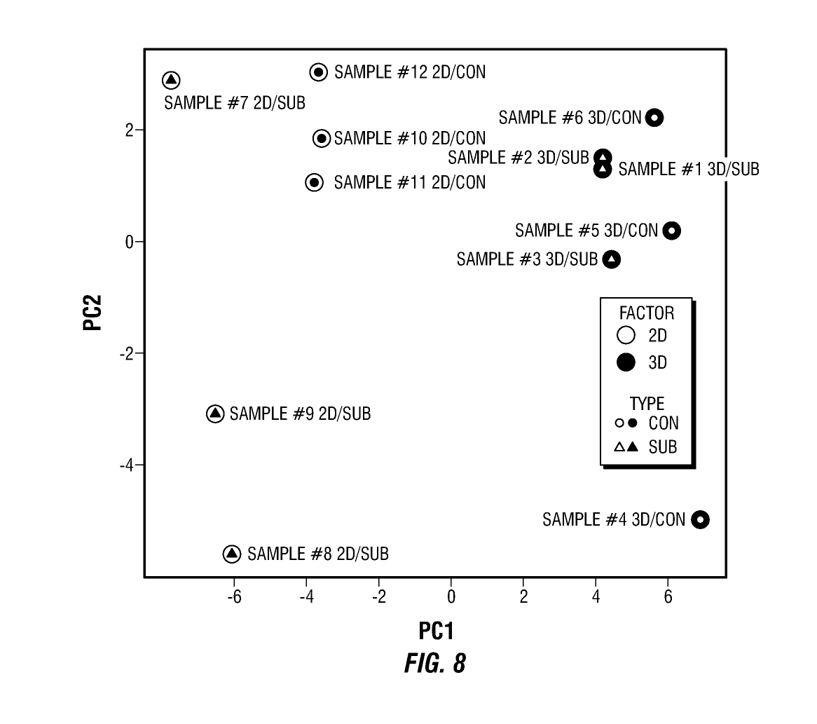

An miRNA screening was performed because it was believed that it would elucidate the behavior of MEECs at the genetic cellular level. Several miRNAs are involved in reducing inflammation and recruiting EPCs. Therefore, the increase of miRNA-126 expression in ECs in a two-dimensional environment and MEECs was measured at different time points, as shown in FIG. 7. miRNA-126 is suppressed by Src, and MEECs are Src-inhibited; therefore, miRNA-126 is upregulated as shown. It should be noted that miRNA-126 overexpression can reduce atherosclerosis, and miRNA-126/126* indirectly suppress inflammatory monocyte recruitment in vivo by downregulating Cc12 in an Sdf-1-dependent manner.

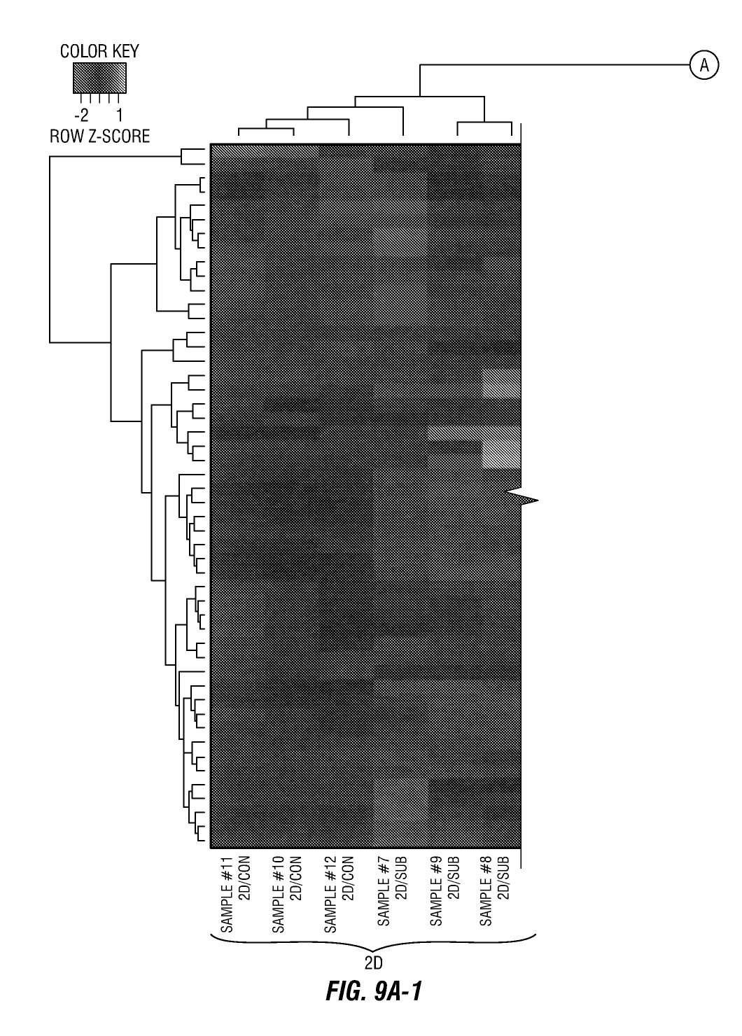

Further testing demonstrated that the EC miRNome depends on the cells' substratum topography. The Principal Component Analysis (PCA) of FIG. 8 presents an overview of the clustering pattern of the samples, which included confluent and subconfluent samples of ECs having 2D and 3D morphologies. FIG. 8 depicts how the samples separated into different regions of the PCA plot based on their biology. The differences were primarily caused by cell substratum and time of culture.

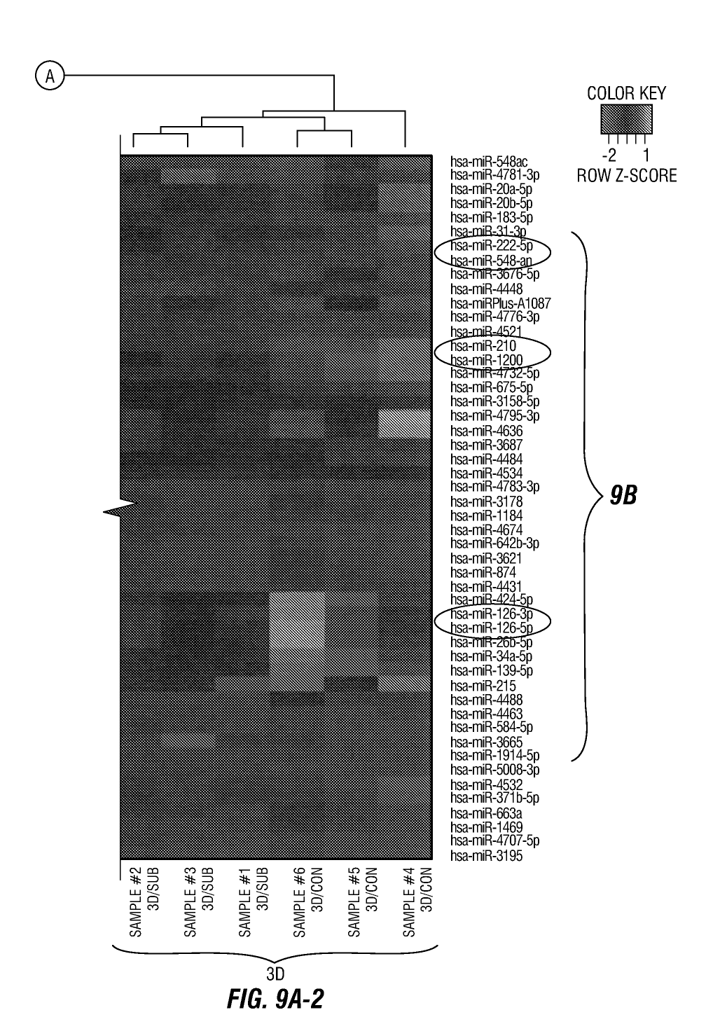

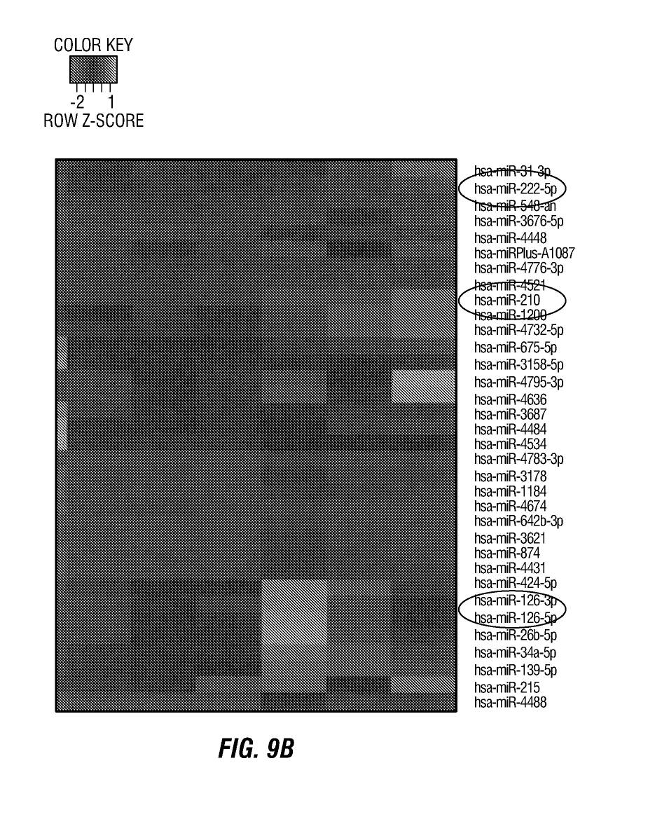

A clustering analysis also was performed on the top 50 microRNAs with the highest standard deviation. This analysis is depicted at FIG. 9, which demonstrates a clear distinction between the ECs having 2D and 3D morphologies. Similarly, FIG. 10 is a volcano plot showing the relation between the logarithm of the p-values and the log fold change between 3D and 2D ECs. The logFC and p-values for three miRNAs believed to be involved in the recruitment of cells as described herein as shown in the following table:

TABLE 1
Selection of interesting miRNA

| miRNA | logFC | p-value | Role/Involvement |
|---|---|---|---|
| miR-210 | 2.281 | 3 × 10−4 | Enhancing cell mediated |
| | | | angiogenesis |
| miR-126 | 0.999 | 2.7 × 10−2 | Important in neovascularization |
| miR-222 | −1.099 | 2 × 10−3 | Negatively modulates angiogenesis |
| | | | by targeting the c-Kit receptor |

### Example 6

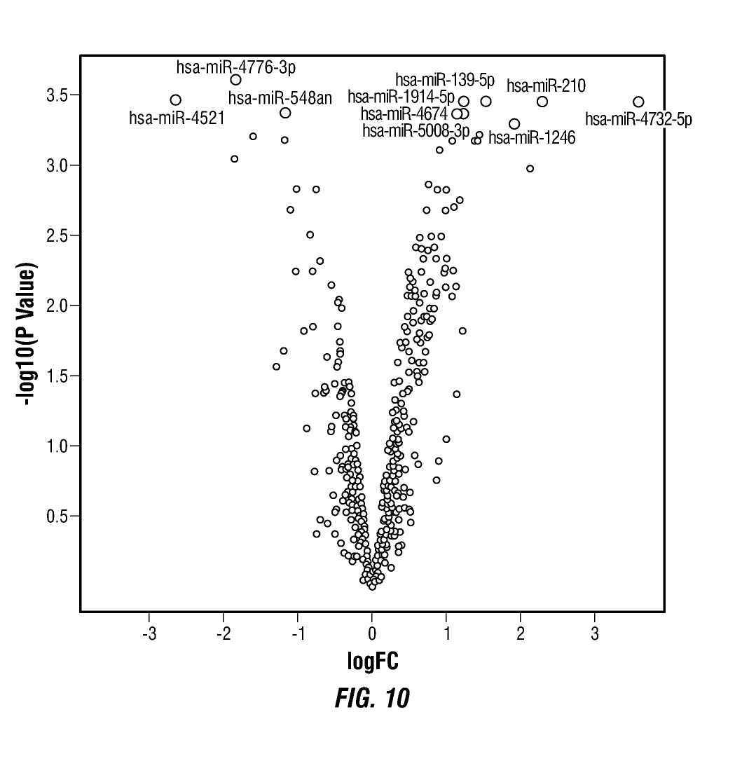

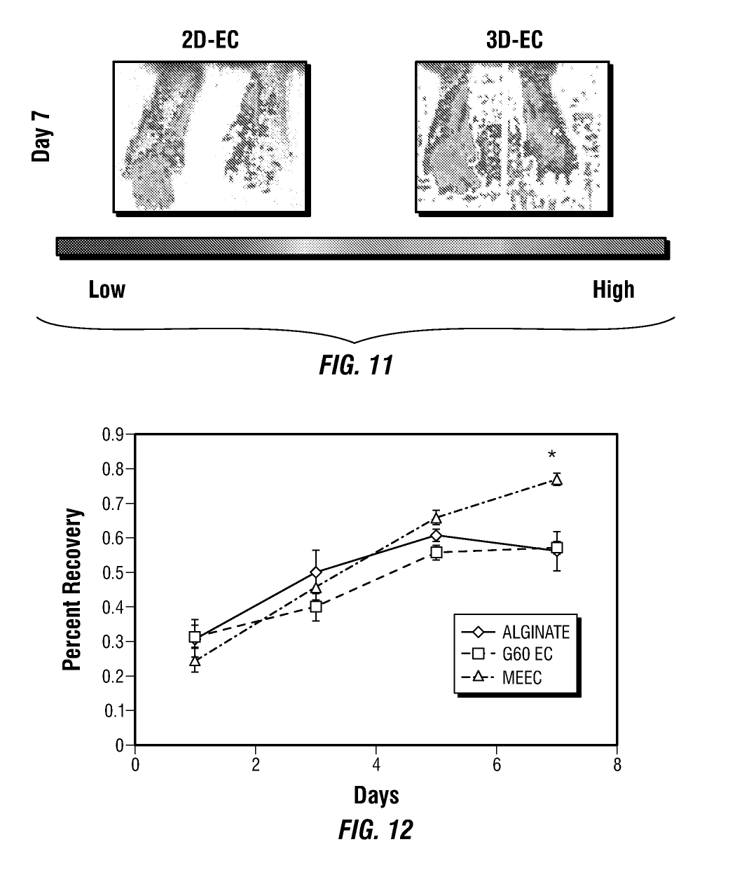

An in vivo functional assay on revascularization was performed. Different media in alginate hydrogel were implanted in mice using femoral artery ligation. Laser speckle imaging was then used to track perfusion, as shown at FIG. 11. FIG. 11 demonstrates that the 3D ECs were more effective than the 2D ECs. In fact, the graph of FIG. 12 demonstrates that the 2D ECs were no more effective at stimulating recovery than the control sample of alginate. The MEECs, however, drastically improved the percent recovery.

Other aspects, embodiments, modifications, and equivalents thereof which, after reading the description herein, may suggest themselves to one of ordinary skill in the art without departing from the spirit of the present invention or the scope of the appended claims.

We claim:

1. A method of making a composition for recruiting progenitor, progenitor-like, or stem cells comprising:
embedding cells in a matrix material having a three-dimensional structure which contacts the cells in a non-planar manner to induce a secretome in an amount effective to recruit progenitor, progenitor-like, or stem cells, wherein the secretome comprises at least one of (i) one or more factors, or (ii) one or more miRNAs.

2. The method of claim 1, wherein the embedded cells comprise endothelial cells, endothelial-like cells, analogs thereof, or a combination thereof.

3. The method of claim 1, wherein the one or more factors comprise SDF-1, HGF, PDGF, TIMP-1, TIMP-2, or a combination thereof.

4. The method of claim 1, wherein the matrix material comprises denatured collagen.

5. The method of claim 1, wherein the matrix material comprises pores having diameters ranging from about 1 μm to about 2000 μm.

6. The method of claim 1, wherein the matrix material comprises struts having dimensions ranging from about 0.01 μm to about 500 μm.

7. A method of recruiting progenitor, progenitor-like, or stem cells to a target site comprising:
positioning at or near the target site a matrix material in which cells are embedded, wherein the matrix embedded cells release at least one of (i) one or more factors, or (ii) one or more miRNAs in an amount effective to recruit progenitor, progenitor-like, or stem cells.

8. The method of claim 7, wherein the target site comprises damaged or diseased tissue in the body of a patient.

9. The method of claim 7, wherein the target site comprises a tumor.

10. The method of claim 7, wherein the embedded cells comprise endothelial cells, endothelial-like cells, analogs thereof, or a combination thereof.

11. The method of claim 7, wherein the one or more factors comprises SDF-1, HGF, PDGF, TIMP-1, TIMP-2, or a combination thereof.

12. The method of claim 7, wherein the matrix material comprises denatured collagen.

13. The method of claim 7, wherein the matrix material comprises pores having diameters ranging from about 1 μm to about 2000 μm.

14. The method of claim 7, wherein the matrix material comprises struts having dimensions ranging from about 0.01 μm to about 500 μm.

15. The method of claim 7, wherein the target site is in a human body.

16. A method of treating a patient comprising:
selecting one or more target sites within a body of a patient in need of treatment; and
implanting in the selected one or more target sites a matrix material in which cells are embedded, wherein the matrix embedded cells release at least one of (i) one or more factors, or (ii) one or more miRNAs in an amount effective to recruit progenitor, progenitor-like, or stem cells to the one or more target sites.

17. The method of claim 16, wherein the one or more target sites comprise a tumor or other damaged or diseased tissue in the body of the patient.

18. A device for use in mobilizing and recruiting progenitor, progenitor-like, or stem cells, the device comprising:
a biocompatible matrix material having a three-dimensional structure; and
cells embedded within the three-dimensional structure of the matrix material, wherein the cells and matrix material are configured to release at least one of (i) one or more factors, or (ii) one or more miRNAs in an amount effective to recruit progenitor, progenitor-like, or stem cells,
wherein the device is in a form suitable for insertion at a target site within a patient in need thereof, the target site comprising a tumor or other damaged or diseased tissue in the patient.

19. The device of claim 18, wherein the embedded cells comprise endothelial cells, endothelial-like cells, analogs thereof, or a combination thereof.

20. The device of claim 18, wherein the form comprises a wrap, foam, gel, particulate formulation, or a combination thereof.

21. The device of claim 18, wherein the device further comprises a pharmaceutically active ingredient or other additive.

22. A method of making a composition for recruiting progenitor, progenitor-like, or stem cells comprising:
embedding cells in a matrix material having a three-dimensional structure which contacts the cells in a non-planar manner to alter the expression of one or more miRNAs in a manner effective to recruit progenitor, progenitor-like, or stem cells.

23. The method of claim 22, wherein the one or more miRNAs comprise miR-210, miR-126, miR-222, or a combination thereof.
